(19) United States
(12) Patent Application Publication
(10) Pub. No.: US 2026/0347296 A1
(43) Pub. Date: Nov. 5, 2026

(54) METHOD FOR THE RECOVERY OF AMYL ALCOHOL

(71) Applicant: ARCHER-DANIELS-MIDLAND COMPANY, Decatur, IL (US)

(72) Inventors: Jose Leboreiro Hernandez, Decatur, IL (US); Joseph Beggin, Warrensburg, IL (US)

(21) Appl. No.: 19/482,451

(22) PCT Filed: May 8, 2023
(86) PCT No.: PCT/US2023/066724

§ 371 Date: Nov. 7, 2025

(51) Int. Cl. C07C 29/80

(52) U.S. Cl. CPC C07C 29/80

(57) ABSTRACT

Methods are provided for the recovery of an amyl alcohol composition comprising 1-pentanol, 2-methyl-1-butanol and 3-methyl-1-butanol from a fusel oil-containing draw from an associated ethanol distillation train, directly and without the use of decantation to concentrate up the fusel alcohols in the fusel oil-containing draw.

### FIELD OF THE INVENTION

The present invention relates to the processing of fusel oils from ethanol production by fermentation.

### BACKGROUND OF THE INVENTION

Fusel oil production is a natural and normal part of the fermentation of sugars by Saccharomyces cerevisiae for producing ethanol. Fusel compounds, however, pose a significant risk to ethanol yield. Both ethanol and fusel compounds are in this regard toxic to yeast growth and fermentation, but fusel compounds are 10 to 15 times more toxic to yeast than is ethanol, and if not properly managed can become part of the process water and recycle to the front end of the plant. A number of instances of fermenters ceasing ethanol production altogether because of fusel oils have been reported.

Fusel compounds are conventionally removed from ethanol plants using a rectifier distillation column, which adds capital, energy and operating cost. By adjusting the pressure, flow and operating temperature in the rectifier column, the fusel compounds are removed from the ethanol via draw points on the side of the column and a fusel draw pump.

This fusel oil-containing draw—and the ethanol fermentation byproducts collectively known as “fusel oil” or equivalently as the“fusel alcohols”—principally comprises a number of three, four and five carbon alcohols, but also includes volatile organic acids, aldehydes, ketones and higher molecular weight fatty acids and esters. Independent of the need to remove these from the ethanol production process for the health of the fermentation, the fusel alcohols individually and/or collectively have economic or market value for application in or for producing solvents, flavors and fragrances, lubricants, adhesives and plasticizers, but their further refining and recovery from ethanol production has heretofore not been economically practicable.

In this regard, the conventional approach to the recovery of fusel alcohols from an ethanol distillation train has involved the use of one or more stages of decantation of the fusel oil draw, whereby large volumes of water are added to cause a phase separation with the upper, lower density phase being enriched with the fusel alcohols. The main problem with this approach is that the water rich phase contains a significant amount of ethanol that must be returned to the ethanol distillation train for recovery. The large volume of added water also creates a large energy load on the process at significant added cost, and the formation of an azeotrope between the water and certain of the fusel alcohols further complicates any effort to recover the fusel alcohols by continuous distillation. Fractional distillation to recover the fusel alcohols in excess of the azeotropic limit is feasible, but costly.

### SUMMARY

In one aspect, the present invention concerns a method for the recovery of fusel alcohols from an associated ethanol distillation train producing a fusel oil-containing draw, without the use of decantation with its associated costs and drawbacks.

In another aspect, the present invention concerns a method for the recovery of fusel alcohols from an associated ethanol distillation train producing a fusel oil-containing draw without the use of decantation to concentrate the fusel alcohols from the fusel oil-containing draw, and for then further refining the recovered fusel alcohols to recover a fusel alcohol product enriched in at least one of the constituent fusel alcohols contained in the fusel oil-containing draw compared to the recovered fusel alcohols from the fusel alcohols-containing draw and to the fusel alcohols-containing draw.

More particularly according to this second aspect, a method is provided for the recovery of an amyl alcohol composition comprising 1-pentanol, 2-methyl-1-butanol and 3-methyl-1-butanol from a fusel oil-containing draw from an associated ethanol distillation train, wherein at least the fusel alcohols from within the fusel oil-containing draw are vaporized to produce a fusel alcohol-containing vapor composition, then the fusel alcohol-containing vapor composition is contacted with a gas-permeable membrane to remove water therefrom and form a dehydrated fusel alcohol-containing vapor composition that can be economically distilled to provide a distillate fraction enriched in an amyl alcohol composition comprising 1-pentanol, 2-methyl-1-butanol and 3-methyl-1-butanol.

In certain embodiments, a portion of the water that has heretofore been removed from a fusel oil-containing draw by decantation is removed by a vapor-liquid separation means (a stripper or distillation column, for example) that also is used to form and forward the fusel alcohol-containing vapor composition, and sufficient additional water is removed through the use of a gas-permeable membrane or series of such membranes to provide a dehydrated fusel alcohol-containing vapor composition that lies to the low water side of the distillation boundary of a ternary phase diagram for the water, ethanol and amyl alcohol system (FIG. 1) and which may consequently undergo simple distillation to provide the distillate fraction enriched in an amyl alcohol composition comprising 1-pentanol, 2-methyl-1-butanol and 3-methyl-1-butanol.

In certain other embodiments, sufficient water is removed from the fusel alcohol-containing vapor composition by means of one or more gas-permeable membranes to provide a dehydrated fusel alcohol-containing vapor composition that lies to the low water side of the distillation boundary of a ternary phase diagram for the water, ethanol and amyl alcohol system, and this composition is again distilled to provide the distillate fraction enriched in an amyl alcohol composition comprising 1-pentanol, 2-methyl-1-butanol and 3-methyl-1-butanol.

These and other aspects, embodiments, and associated advantages will become apparent from the following Detailed Description.

### BRIEF DESCRIPTION OF THE DRAWINGS

FIG. 1 is a ternary phase diagram for the distillation of a mixture of water, ethanol and 3-methyl-1-butanol in various proportions.

FIG. 2 is a schematic illustration of a method for the recovery of an amyl alcohol composition in an illustrative embodiment.

### DETAILED DESCRIPTION OF EMBODIMENTS

The disclosures of all patent and non-patent literature referenced herein are hereby incorporated in their entireties.

As used in this application, the singular forms “a”, “an” and “the” include plural references unless the context clearly indicates otherwise. The term “comprising” and its derivatives, as used herein, are similarly intended to be open ended terms that specify the presence of the stated features, elements, components, groups, integers, and/or steps, but do not exclude the presence of other unstated features, elements, components, groups, integers and/or steps. This understanding also applies to words having similar meanings, such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers, and/or steps. The term “consisting essentially of”, as used herein, is intended to specify the presence of the stated features, elements, components, groups, integers, and/or steps, as well as those that do not materially affect the basic and novel characteristic(s) of stated features, elements, components, groups, integers, and/or steps.

Terms of degree such as “substantially”, “about” and “approximately” as used herein mean a reasonable amount of deviation of the modified term (beyond that degree of deviation understood by the precision (significant figures) with which a quantity is expressed) such that the end result is not significantly changed. These terms of degree should be construed as including a deviation of at least plus or minus five (5) percent from the stated value, provided this deviation would not negate the meaning of the term modified.

The term “biologically-derived” as used herein is used interchangeably with “biobased” or “bioderived”, and “biologically-derived”, “biobased” and “bioderived” shall all be understood as referring to any chemical compounds, including monomers and polymers, that are obtained, in whole or in any part, from any renewable resources including but not limited to plant, animal, marine materials or forestry materials. The “biobased content” of any such compound shall be understood as the percentage of a compound's carbon content determined to have been obtained or derived from such renewable resources by ASTM Method D6866. In this regard ASTM Method D6866, similar to radiocarbon dating, compares how much of a decaying carbon isotope remains in a sample to how much would be in the same sample if it were made of entirely recently grown materials. Samples are combusted in a quartz sample tube and the gaseous combustion products are transferred to a borosilicate break seal tube. In one method, liquid scintillation is used to count the relative amounts of carbon isotopes in the carbon dioxide in the gaseous combustion products. In a second method, 13C/12C and 14C/12C isotope ratios are counted (14C) and measured (13C/12C) using accelerator mass spectrometry. Zero percent 14C indicates the entire lack of 14C atoms in a material, thus indicating a fossil (for example, petroleum based) carbon source. One hundred percent 14C, after correction for the post-1950 bomb injection of 14C into the atmosphere, indicates a modern carbon source. ASTM D6866 effectively distinguishes between biobased materials and petroleum derived materials in part because isotopic fractionation due to physiological processes, such as, for example, carbon dioxide transport within plants during photosynthesis, leads to specific isotopic ratios in natural or biobased compounds. By contrast, the 13C/12C carbon isotopic ratio of petroleum and petroleum derived products is different from the isotopic ratios in natural or bioderived compounds due to different chemical processes and isotopic fractionation during the generation of petroleum. In addition, radioactive decay of the unstable 14C carbon radioisotope leads to different isotope ratios first in biobased products compared to petroleum products.

The present invention may be more completely understood by describing certain embodiments in greater detail. These embodiments are not to be taken as limiting the scope and breadth of the current invention as more particularly defined in the claims that follow, but are illustrative of the principles behind the invention and demonstrate various ways and options for how those principles can be applied in carrying out the invention.

Thus, unless otherwise indicated, any definitions or embodiments described in this or in other sections are intended to be applicable to all embodiments and aspects of the subjects herein described for which they would be suitable according to the understanding of a person of ordinary skill in the art.

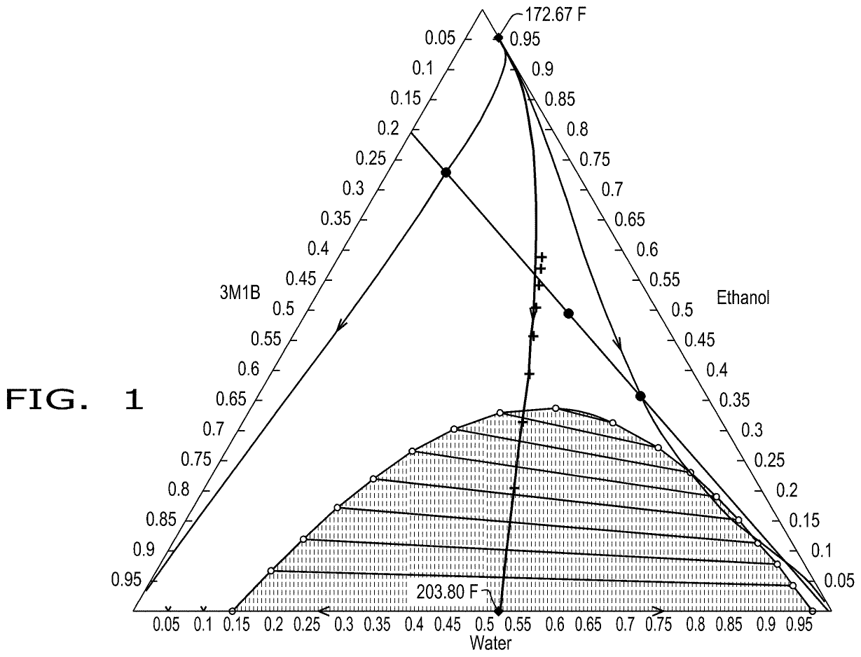

Turning now to FIG. 1, a ternary phase diagram is shown for the distillation of various combinations of water, ethanol and 3-methyl-1-butanol, one of the amyl alcohols of interest for purposes of the present invention. As those of skill in the art will appreciate, mixtures to the left, low water side of the illustrated distillation boundary may more readily and easily be distilled to provide the desired distillate fraction enriched in the amyl alcohols, so in preferred embodiments the method of the present invention seeks to achieve such a mixture from a fusel oil-containing draw, fundamentally either by a) a combination of removing a portion of water in forming a fusel alcohols-containing vapor composition directly from a fusel oil-containing draw, and then passing that fusel alcohols-containing composition into contact with one or more gas-permeable membranes which remove the remainder of the water necessary to achieve a further dehydrated composition lying to the left of the distillation boundary or b) forming the fusel alcohols-containing vapor composition and removing all of the water necessary to achieve a composition lying to the left of the distillation boundary by means of one or more gas-permeable membranes.

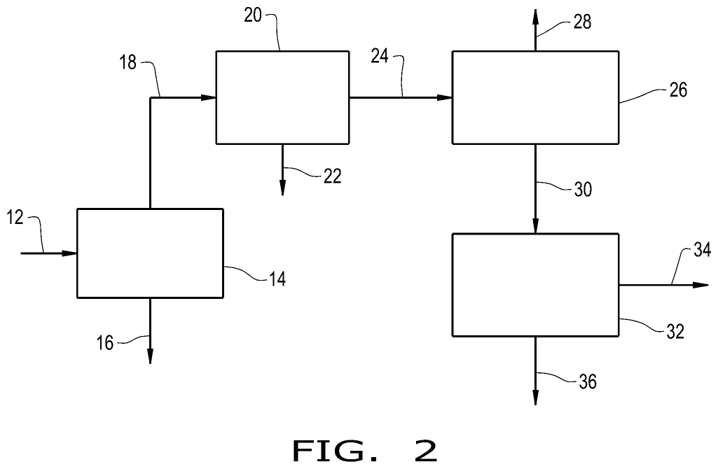

Turning now to a consideration of FIG. 2, an illustrative embodiment 10 is schematically shown of a method according to the present invention according to the first of the aforementioned approaches, whereby a fusel oil-containing draw from the distillation section of a production plant for producing ethanol by the fermentation of sugars is refined to provide an amyl alcohol composition comprising 1-pentanol, 2-methyl-1-butanol and 3-methyl-1-butanol without the necessity of using any decantation.

The fusel oil-containing draw 12 enters a single stage vaporizer 14 to separate a substantial portion of the water contained in the fusel oil-containing draw together with some ethanol, in a liquid fraction 16 that can be recycled to the distillation section of the associated ethanol production plant for recovering the ethanol therein, from a water-depleted fusel alcohol-containing vapor composition 18 that is brought into contact with a gas-permeable membrane or series of such membranes 20 that removes (or that remove) additional water in stream 22 from the water-depleted fusel alcohol-containing vapor composition, to form a further dehydrated fusel alcohol-containing vapor composition 24 in which water is preferably less than about 25 percent by weight of the composition.

A preferred membrane 20 for our purposes would be a zeolite film membrane on a porous ceramic support, in particular, a zeolite film membrane on a porous tubular ceramic support in a modular bundle of such tubular membranes in a tube-and-shell configuration. Commercial exemplars are currently sold for use in ethanol dehydration by a number of manufacturers, for example, by Mitsubishi Chemical Engineering Corporation, Tokyo, Japan and Hitachi Zosen Corporation, Osaka, Japan. Especially preferred are the CHA-, MOR- and A-type porous alumina supported tubular zeolite membranes from Hitachi Zosen. Other, non-zeolitic membranes such as the hollow fiber polymeric membranes sold by Whitefox Technologies, London, England may also be useful, to the extent the same are robust enough to accommodate the vapor phase dehydration of water-depleted fusel alcohol-containing vapor composition 18 under conditions needed to avoid condensation throughout due to heat losses and changes in the water content of the composition 18 as the same passes cross-currently over or counter-or concurrently over and along the membrane 20 and is progressively dehydrated. Consequently, some degree of superheating of the water-depleted fusel alcohol-containing vapor composition 18 will be required, though with this qualification, the process 10 is amenable to a range of operating pressures and temperatures consistent with the selection of a particular membrane or combination of membranes 20. Combinations of the same or of different membranes 20 may in this regard be employed in series, operating under the same or different conditions—for example, ceramic-supported zeolitic membranes of the MOR and A-types in series—or combinations of the same or different membranes 20 may be employed in parallel modules for facilitating maintenance. The selection of a particular configuration, membrane material and appropriate operating conditions will be well within the capabilities of those of skill in the art.

The further dehydrated fusel alcohol-containing vapor composition 24 is then condensed and supplied to a first distillation column 26, producing an overhead stream 28 comprised of any residual water and more volatile alcohols such as the C2-C4 alcohols, and a bottoms stream 30 comprised of the Cs alcohols and heavier, higher distilling constituents of the fusel alcohol-containing vapor composition 24 is further distilled in a second distillation column 32. Second distillation column provides a distillate fraction enriched in an amyl alcohol composition 34 comprising 1-pentanol, 2-methyl-1-butanol and 3-methyl-1-butanol while residual heavier compounds such as fatty acid ethyl esters, phenylethyl alcohol and phenylethyl acetate exit the second distillation column 32 in bottoms stream 36.

The amyl alcohol composition 34 may in certain embodiments then be passed in a further step (not shown in FIG. 2) into contact with a suitable adsorbent, for example, an activated carbon, for removing any trace impurities that may be present in the composition 34 and that would be deleterious or unwanted in contemplated applications or uses of the amyl alcohol composition 34 or any component thereof that those of skill in the art may wish to further isolate and refine.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method for the recovery of fusel alcohols from a fusel oil-containing draw from the distillation of an ethanol fermentation product mixture, comprising:
vaporizing at least fusel alcohols from within the fusel oil-containing draw to produce a fusel alcohol-containing vapor composition;
optionally, concurrently removing at least some water from the fusel-oil containing draw in the vaporizing step; and
contacting the fusel alcohol-containing vapor composition with one or more gas-permeable membranes to remove water from the fusel alcohol-containing vapor composition,
whereby, by means of the one or more gas-permeable membranes alone or by means of the one or more membranes acting in combination with the optional removal of water in the vaporizing step, a sufficiently dehydrated fusel alcohol-containing composition results which lies to the left, low water side of the distillation boundary of a ternary phase diagram for the distillation of various ethanol, water and amyl alcohol mixtures.

2. The method of claim 1, wherein by means of the one or more gas-permeable membranes alone or in combination with the removal of water in the vaporizing step, a fusel alcohol-containing composition containing less than about 25 percent by weight of water is achieved.

3. The method of claim 2, further comprising distilling the fusel alcohol-containing composition to provide a distillate fraction enriched in an amyl alcohol composition comprising 1-pentanol, 2-methyl-1-butanol and 3-methyl-1-butanol.

4. The method of claim 3, further comprising contacting the distillate fraction enriched in the amyl alcohol composition with a carbon adsorbent.

5. A method for the recovery of amyl alcohol from a fusel oil-containing draw from the distillation of an ethanol fermentation product mixture, comprising:
obtaining the fusel oil-containing draw;
directly supplying the fusel oil-containing draw to a vapor-liquid separation means for vaporizing at least fusel alcohols from within the fusel oil-containing draw and producing a fusel alcohol-containing vapor composition;
contacting the fusel alcohol-containing vapor composition with one or more gas-permeable membranes to remove water from the fusel alcohol-containing vapor composition, to form a dehydrated fusel alcohol-containing vapor composition; and
carrying out fractional distillation of the dehydrated fusel alcohol-containing vapor composition to provide a distillate fraction enriched in an amyl alcohol composition comprising 1-pentanol, 2-methyl-1-butanol and 3-methyl-1-butanol.

6. The method of claim 5, further comprising contacting the distillate fraction enriched in the amyl alcohol composition with a carbon adsorbent.
